(19) United States
(12) Patent Application Publication
(10) Pub. No.: US 2023/0225834 A1
(43) Pub. Date: Jul. 20, 2023

(54) REMOVABLE AIRWAY APPLIANCE

(71) Applicant: Nathan Milo Tanner, B Medford, OR (US)

(72) Inventor: Nathan Milo Tanner, B Medford, OR (US)

(21) Appl. No.: 18/154,996

(22) Filed: Jan. 16, 2023

Related U.S. Application Data
(60) Provisional application No. 63/300,090, filed on Jan. 17, 2022.

(51) Int. Cl. A61C 7/08; A61C 7/36; A61M 16/00

(52) U.S. Cl. CPC A61C 7/08; A61C 7/36; A61M 16/00

(57) ABSTRACT

The present disclosure provides removable airway appliance for guiding and developing the lower and upper jaws forward. The present invention prevents crowding of the teeths and makes room for all the teeth, making room for all permanent teeth without crowding, creates space for all the permanent teeth. The present invention may grow the upper jaw forward and unrestrict the lower jaw that is often trapped back behind the upper jaw. The present invention guides and develops the lower and upper jaws forward, and widen the upper jaw, making room for the tongue in the mouth to maximize the airway space. In removable airway appliance, on the horizontal axis the screws can be positioned flat at a small angle or at a steeper angle to help direct the direction of growth and development of the upper and lower jawbones.

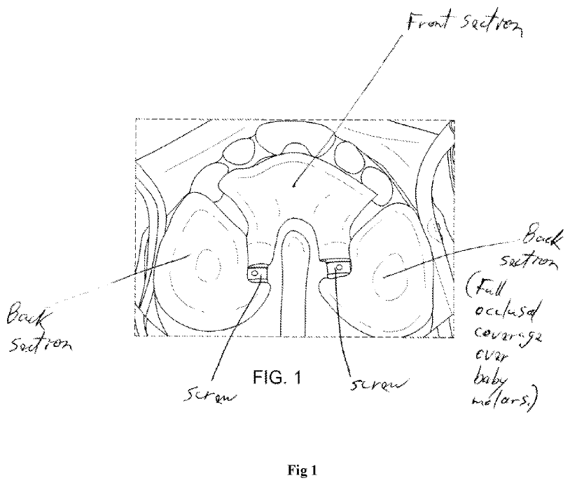

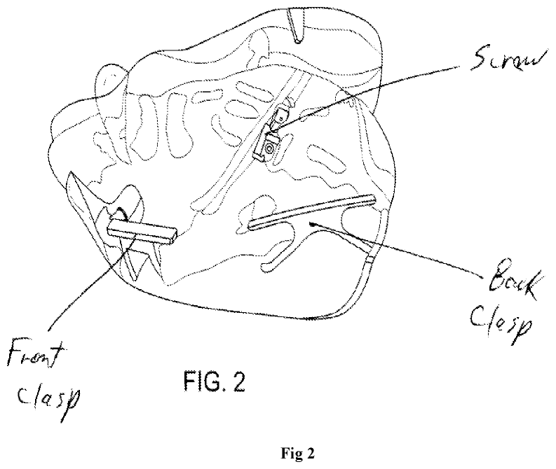

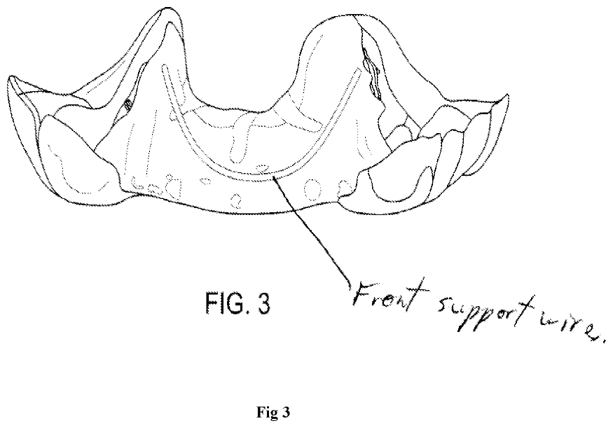

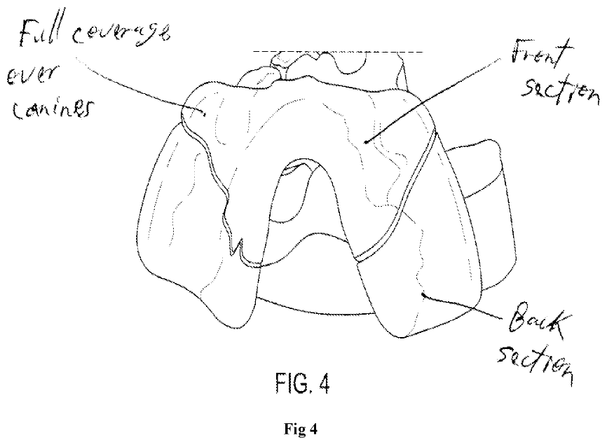

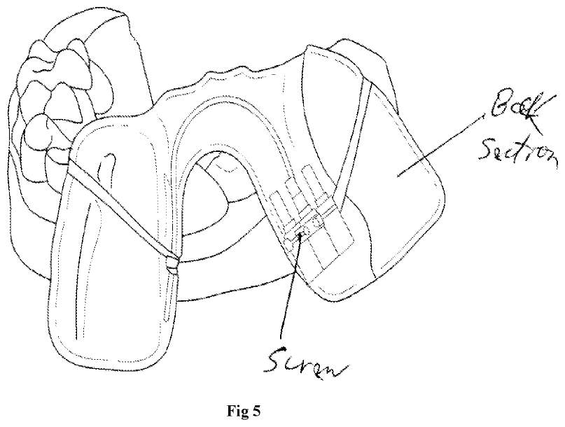

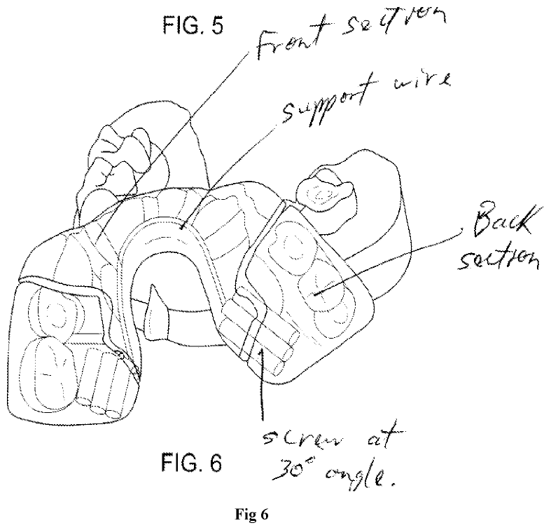

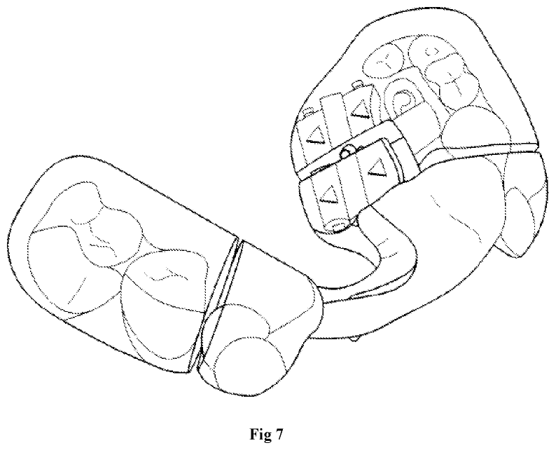

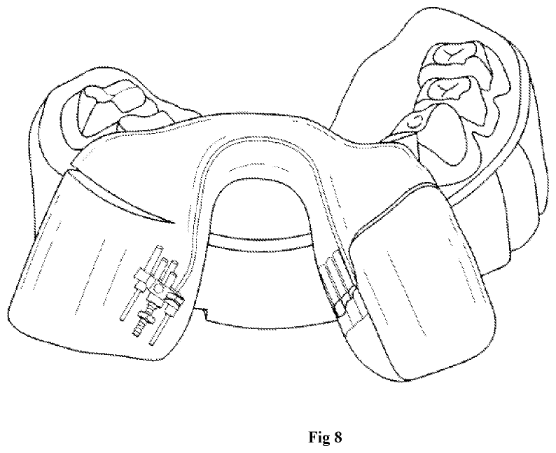

### FIELD OF THE DISCLOSURE

The present disclosure relates generally to a removable airway appliance. More specifically, and without limitation, the present disclosure relates to a removable airway appliance for developing the bone of the upper and lower jaws forward permanently. However, the present disclosure is not limited to these novel and inventive systems and improvements and methods of use, and it may further be adapted for a variety of purposes.

### BACKGROUND OF THE DISCLOSURE

Sleep disorders are a group of conditions that affect the ability to sleep well on a regular basis. Sleep disorders are caused by too much soft tissue in the throat. Most people occasionally experience sleeping problems due to stress, hectic schedules, and other outside influences. However, when these issues begin to occur on a regular basis and interfere with daily life, they may indicate a sleeping disorder. Depending on the type of sleep disorder, people may have a difficult time falling asleep and may feel extremely tired throughout the day. The lack of sleep can have a negative impact on energy, mood, concentration, and overall health. In some cases, sleep disorders can be a symptom of another medical or mental health condition. These sleeping problems may eventually go away once treatment is obtained for the underlying cause. When sleep disorders aren't caused by another condition, treatment normally involves a combination of medical treatments and lifestyle changes.

There are many different types of sleep disorders like Insomnia, Sleep apnea, Parasomnias, Restless leg syndrome, Narcolepsy, and (UARS) Upper Airway Respiratory Syndrome. Some may be caused by other underlying health conditions. Sleep apnea is a common sleep disorder. It occurs when the muscles in the back of the person's throat relax and then narrow or close the airway. With the tissue blocking the air passage, the person can't get air in, and air can't get out. During sleep apnea, the person repeatedly stops breathing during sleep. The person will briefly wake up to resume breathing, even if that particular person isn't aware of it. The interrupted sleep can lead to symptoms such as: excessive daytime sleepiness; snoring; sleep deprivation; insomnia; dry mouth; headache.

Sleep apnea is a potentially serious sleep disorder in which breathing repeatedly stops and starts. Snoring, upper airway resistance syndrome and obstructive sleep apnea syndrome (OSAS) are all related to narrowing or obstruction of the upper airway during sleep (sleep disordered breathing). According to the national institutes of health (NIH), approximately 18 million. Americans have sleep apnea (sleep disordered breathing), but fewer than 50% are presently being diagnosed. According to the national highway traffic and safety administration (NHTSA), 100,000 accidents and 1,500 traffic fatalities per year are related to drowsy driving. More than 50 percent, of Americans over age 65 have sleep difficulties, and prevalence of sleep problems will therefore increase as the over-65 population increases. Each year, sleep disorders, sleep deprivation, and excessive daytime sleepiness add approximately $16 billion annually to the cost of health care in the U.S. and result in $50 billion annually in lost productivity.

The upper airway (air filled space between the nose and larynx) of the human beings is curvy, so it is more flexible than that of other species and is more prone to collapse under negative pressure. The air cavity at the back of the throat is partly responsible for sleep disorders. When human beings are awake, a certain amount of tone is present in upper airway muscles to prevent this collapse. During sleep time, muscle tone decreases in upper airway muscles and in this way, in some individuals, this causes the airway to collapse.

A small amount of decreased airflow or brief obstructions occur in all humans during sleep. They are counted as medically significant if airflow is decreased more than 50% of normal (hypopnea) or if airflow is obstructed for more than 10 seconds (apnea) for the severity of the sleep disorder. These repetitions of hypopnea or apnea cause some degree of arousal during sleep. The patient is not completely awakened to full consciousness, the sleep pattern is disturbed causing the patient to feel sleepy during the day. If the frequency of hypopnea or apnea is less than 5 times an hour it is called upper airway resistance syndrome. These patients often show symptoms related to sleep disruption. If the frequency is more than 5 times an hour it is sleep apnea.

Various experimentations have been carried out for expansion of the upper and lower jaws in a forward direction. In the 1950's Dr. John Mew In England started using orthotropic using a bio block stage 1 to expand the upper jaw and a Bio Block 2 to force the lower jaw forward. The upper teeth would be flared out and the lower jaw was forced forward with the bio block stage 2, and in 1980's and 90's Dr. Bill Hang in California developed his own practices that are improved on the Mew invention, he calls it an Expanser on the top, it also flares the upper teeth, and he calls the lower appliance the Adapt Appliance and it forces the lower jaw forward. Dr. Bill Hang also does reopening extraction spaces to make more room for the tongue in the mouth. Dr. Steve Galella uses a fixed and a removable Anterior Growth Guidance appliance or Osseous Restoration Appliance to guide forward development. These are two other products that develop the upper and lower jaws forward.

There are various Mandibular (jaw) advancement devices (MAD's) and jaw development devices. The Adjustable PM Positioner was invented by Dr. Jonathan A. Parker. Its unique design is durable, comfortable, and effective. The Adjustable PM Positioner is constructed of a heat-sensitive acrylic (Bruxeze) that fits comfortably over the upper and lower teeth. The appliance stabilizes the jaw position by allowing only a small amount of jaw movement. The adjustment mechanisms are on the cheek side of the appliance and allow for easy advancement of the jaw position to improve effectiveness of the device when it is indicated. It is a durable and effective option.

One of the prior art means is disclosed in patent number 20210007831 as “A Dental system for symmetry of jaw, palate and teeth”. A dental apparatus, systems and methods for providing anterior, lateral and vertical movement, an apparatus including a posterior portion to be fixed to and supported by a posterior dental structure of a patient; and an anterior portion connected to and separable from the posterior portion, the anterior portion operable to provide anterior movement, lateral movement and vertical movement to an anterior dental structure of the patient. The dental system include screws that can be positioned anywhere in the device for controlled movement in multiple directions (planes) for dental structure movement in adults and children. The adjustable dental expander devices provide anterior, vertical, and/or anterior-lateral movement to teeth, a jawline, and/or palate. The devices are compact and reduce bulkiness and provide a soft surface to reduce direct pressure and allow for 24-hour wear and slow controlled movement. The devices do not impede speech while worn. One or more surfaces of the device is optionally covered with a thin cover or coating of soft, gel like material to distribute pressure and guard against painful pressure points. A suitable gel-like material is soft to prevent undue pressure on any particular point, but provides gentle pressure over time at particular points.

The device comprises of a hinge and a wire in between first anterior portion and first posterior portion and a second adjustable and lockable hinge is present in between second anterior portion and second posterior portion. The device includes a screw base and a screw which is operable to allow rotation in and out of the screw base such that the rotation provides movement and pressure in the anterior dental structure. The device includes one or more attachable pads positioned on the anterior portion of the device which provides movement and pressure to the anterior dental structure. Dental device can be inserted to be made semi-permanent via dental cementation or glue, or it can be removable and re-insert able by the patient and the doctor. The device can be applied for movement in the upper portions and lower portions of a patient's mouth to apply movement to upper and lower jaw structure.

The posterior and anterior portion are connected by a wire, which is operable to move in an anterior direction and hinge is allowing rotation of the anterior portion.

Another one prior art means is disclosed in patent no. 20200214804 as Orthodontic appliance. The present invention shows a jaw shape modifying device comprising a frontal shell portion and lateral shell portions connected by adjustment mechanisms for adjusting the frontal distance between the frontal shell portion and the lateral shell portions. It comprises of a shell shaped structure which includes a shape-biasing region which extends beyond the patient's internal gum line when the shell is worn. The appliance reduces the severity of orthodontic relapse, thereby achieving a more stable outcome for the patient.

The shell includes a front shell secured to lateral shell portions by way of an adjustment mechanism for adjusting a distance between the frontal shell portion and lateral shell portions by way of an adjustment mechanism for adjusting a distance between the frontal shell portions and the lateral shell portions. The forward region of the patient's jaw is extended by increasing the frontal distance over time. The adjustment mechanism includes an expansion screw securable to the jaw biasing region of each shell portion. The shell portions are interconnected to one another by one or more expansion screws which are easy to operate.

The appliance comprises one or more anchor points to which a resilient band is attachable. An anchor point could be a hook embedded into the appliance. The first appliance is configured to be worn over the patient's upper teeth, and the second appliance is configured to be worn over the patient's lower teeth. The orthodontic appliances modify the shape of one or more jaws individually but also work to alter the position of one jaw relative to the other.

Another prior art means is disclosed in U.S. Pat. No. 5,002,485 as “orthopedic appliance”. It shows an orthopedic appliance having a universal screw assembly that interconnects frontal portions and the first and second side portions and operates to independently adjust the lateral spacing of the side portions from one another and the anterior-posterior spacing of the frontal portion from the side portions. The present invention is an orthopedic appliance for correcting class II malocclusions comprises a frontal portion to engage the mandibular and the maxillary frontal arches and first and second side portions, posterior to the frontal portion. Configured to engage at least some of the maxillary molars. A universal screw assembly interconnects the frontal portions and the first and second side portions and operate to adjust the lateral spacing of the side portions from one another and the anterior posterior spacing of the frontal portion from the side portions. The appliance is expanded in stages to maximize the utilization of corrective lower jaw movements which results from securing the appliance in patient's upper mouth.

Another prior art means is disclosed in U.S. Pat. No. 4,433,956 as “Orthopedic corrector and method of correction of class II malocclusion”. The orthopedic appliance is designed so that when the posterior and anterior segments are minimally expanded, the mandibular-maxilla position is changed from which exists prior to treatment. As time progresses, the expansion screw assemnblies are turned , separating the anterior and posterior segments with resulatnt movement of the mandible more forward. The orthopedic corrector of the appliance is outfitted with orthodontic attachments such as a labial archwire. An improved orthopedic appliance for correcting a class II malocclusion comprising an acrylic anterior segment molded to fit the lower mouth and dentition and two acrylic posterior segments molded to fit upper mouth and dentition of a patient and an expansion screw connecting each posterior segment to the anterior segment for expandiable movement between the anterior segment and the posterior segments. The applaianace is expanded in stages to maximize the utilization of corrective lower jaw movements which results from the anchoring of the orthopedic appliance in the patient's upper mouth.

Another one prior art means is disclosed in U.S. Pat. No. 4,431,411 as “Segmented labial bow with lip pads for aid in the correction of class III malocclusions and malocclusions with underdeveloped maxillas”. The segmented labial bow with lip pads is attcahed to a conventional orthopedic/orthodontic appliance. As the orthopedic/orthodontic appliance is activated, the lip pads on the segmented bow hold the upper lip away from the roots of the maxillary anterior teeth and alveolar process allowing skeletal development of the premaxillary area with resulatant forward movement of the roots and alveolar bone of the premaxillary teeth. It shows an acrylic orthopedic/orthodontic appliance having clasps 12, 15 for attachment to the teeth and jackscrews 16 that cause the premaxilla to move forward in relation to the mandible.

The segmented bows are attached to the sides of the active orthopedic appliance with solder on a wire originating in the acrylic portion of the appliance.

Another prior art means is disclosed in KR 20210008908 as mouthpiece. There is provided a mouthpiece, characterized in that the divided pieces are overlapped so that a surface that faces or comes into contact with the skin of the oral cavity does not form a gap despite the relative movement or approach of the divided pieces.

The mouthpiece of the present invention for solving the above problems is mounted in the oral cavity and used to deform the shape of the skull including the maxilla, and consists of a plurality of divided pieces, at least some of the divided pieces are formed by a screw. In the mouthpiece configured to be approached and spaced from each other, the divided pieces are overlapped so that the surfaces of the divided pieces that face or come into contact with the skin in the oral cavity do not form a gap despite the relative movement or approach of the divided pieces. It is mounted in the oral cavity and used to modify the shape of the skull including the maxilla, and is composed of a plurality of divided pieces, wherein at least some of the divided pieces are configured to be approached and spaced apart from each other by a screw, the mouthpiece comprising: At least some of the dividing pieces are connected to a headgear mounted outside the oral cavity, and the reaction force vector with respect to the action force vector by the screw is set to be offset by the support force vector by the headgear.

Or, it is mounted in the oral cavity and used to modify the shape of the skull including the maxilla, and is composed of a plurality of divided pieces, wherein at least some of the divided pieces are configured to be approached and spaced apart from each other by a screw, the mouthpiece comprising: At least some of the split pieces are connected to a connecting wire, the connecting wire is connected to a headgear mounted outside the oral cavity, and a blind hole or a through hole is formed in the latch on the lower surface of the split piece connected to the connecting wire. The hook of the connecting wire is characterized in that it is provided with a projection (positive shape) that is detachably coupled to the blind hole or the through hole.

Or, it is mounted in the oral cavity and used to modify the shape of the skull including the maxilla, and is composed of a plurality of divided pieces, wherein at least some of the divided pieces are configured to be approached and spaced apart from each other by a screw, the mouthpiece comprising: At least one of the divided pieces is preferably provided with a power storage device for storing electrical energy or a power generation device for converting the oral masticatory force into electrical energy by the piezoelectric phenomenon.

It is characterized in that it is made of a material having a surface roughness Ra of 0.2 to 0.4 so as not to form a bacterial film.

Another prior art means is disclosed in KR 970010577 as device for jaw orthopedic. The han appliance fundamentally treats the third class malocclusion of jaw bones without any operation by effectively utilizing the period of most active growth orthopedically to induce the proper growth of the bones. The construction includes a premaxillae (500), an orthopedic part (100) tightly attached to the inner side of a premaxillae (500), q fixation part (200) fixed to the inner side of the upper jaw molar teeth and palatal surface, the parts (100) and (200) having a jack screw (300) installed between them, the fixation part (200) being connected to a lower jaw holder (400) on the inner side of the lower jaw bone to be integral, the fixation part (200) and the holder (40) having a protrusive bite block (30) for pressure-attaching the lower and upper molar teeth (502, 602) on both sides of the loader center. The holder (40) having a labial bow (41) on its tip to fix the lower anterior teeth.

Another prior art means is disclosed in WO2021049673 as a “Mouthpiece”. Provided is a mouthpiece which is mounted in an oral cavity for use in changing the shape of the skull including the maxillary bone and is composed of a plurality of segment pieces, at least some of the segment pieces being configured to approach or be spaced apart from each other by means of a screw, wherein the surfaces of the segment pieces facing or contacting the skin in the oral cavity are configured such that the segment pieces overlap with each other, such that a gap does not occur in spite of the approaching or spacing-apart movement of the segment pieces relative to each other.

Or, it is mounted in the oral cavity and used to modify the shape of the skull including the maxilla, and is composed of a plurality of divided pieces, wherein at least some of the divided pieces are configured to be approached and spaced apart from each other by a screw, the mouthpiece comprising: At least some of the dividing pieces are connected to a headgear mounted outside the oral cavity, and a reaction force vector Δα with respect to an action force vector a by the screw is set to be offset by a support force vector Δϕ by the headgear.

Or, it is mourned in the oral cavity and used to modify the shape of the skull including the maxilla, and is composed of a plurality of divided pieces, wherein at least sonic of the divided pieces are configured to be approached and spaced apart from each other by a screw, the mouthpiece comprising: At least some of the divided piece, the connecting wire is connected to the connecting wire is connected to a headgear mounted in addition to the oral cavity, the latch of the lower face of the split pieces are connected to the connecting wire, blind holes or through holes are formed, The hook of the connecting wire is characterized in that it is provided with a projection (positive shape) that is detachably coupled to the blind hole or the through hole.

Or, it is mounted in the oral cavity and used to modify the shape of the skull including the maxilla, and is composed of a plurality of divided pieces, wherein at least sonic of the divided pieces are configured to be approached and spaced apart from each other by a screw, the mouthpiece comprising: At least one of the divided pieces is preferably provided with a power storage device for storing electrical energy or a power generation device for converting the oral masticatory force into electrical energy by the piezoelectric phenomenon.

At this time, it is preferable that at least one of a low-frequency generator, an infrared LED, an ultraviolet LED, a screw driving motor, a wireless communication module with an external terminal, and a screw operation amount sensor is driven by the electric energy.

In addition, it is characterized in that it is made of a material having a surface roughness Ra of 0.2 to 0.4 so as not to form a bacterial film.

Another prior art means is disclosed in patent no: EP2067452 as “Orthodontic device”. Orthodontic device (1) for activating the growth of the lower jaw for use in combination with fixed orthodontic appliances, comprising two plastic bodies (2,2′), each provided with at least one counter-surface corresponding to the chewing surface of molars in the upper jaw (6,6′), and at least one counter-surface corresponding to the chewing surface of molars in the lower jaw (7,7′), and further attaching means (8, 8′, 9 , 9′) for attaching to the upper jaw, characterized in that each plastic body (2, 2′) comprises at least two parts ((4, 5) , (4′, 5′)) which can be displaced and fixed relative to each other substantially parallel to the longitudinal direction of the respective counter-surfaces, wherein each part is provided with at least one of said counter-surfaces.

Dorsal (Somnodent)

The Dorsal appliance can be made of a hard acrylic or an acrylic material that has a softer inner lining It is specifically designed with separate upper and lower portions for easier placement and removal. There is a wing on the lower portion and an acrylic block on the upper portion that encases a screw mechanism for easy advancement. This is a good choice for patients with smaller mouths or narrower dental arches and is a comfortable and effective choice for sleep apnea.

Dream TAP

The Dream TAP is made of a material that has a hard outer shell and softer inner lining It has a hook and screw on the front of the lower portion and the hook attaches to a wire on the upper portion that connects the appliance together. Since the screw mechanism is on the front of the device, the appliance can be advanced while in the mouth by using a small key. It is durable and effective in treating sleep apnea.

EMA

The EMA is made of thinner acrylic material that fits over the teeth and the upper and lower portions are connected by flexible straps. Straps of different lengths and flexibilities allow for advancing the jaw position gradually and they allow freedom of movement of the jaw. This device is used in patients with primary snoring and milder sleep apnea conditions.

Herbst

The Herbst appliance is made of either a heat-sensitive or hard acrylic material and has a piston-type (post within a tube) screw mechanism along the sides of the device. It allows good freedom of movement of the jaw in all directions. It is durable and effective in treating sleep apnea.

Micro2

The Micro2 is an all-acrylic (no metal) appliance that has wings on the upper and lower portions that are positioned against each other to hold the lower jaw forward. The design provides a lower profile and allows for good jaw movement. The patient receives 4-6 units that have the wings positioned in a way that allows for mixing and matching these units to gradually advance the jaw position. This device is an effective option for treating sleep apnea.

Narval

The Narval is made of a nylon material that allows it to be one of the thinnest and lightest appliances available. It has plastic straps that connect the upper and lower portions and has no metal components. It allows for good jaw movement, is effective for treating sleep apnea, and is a good option for patients who have a smaller mouth.

SUAD

The SUAD appliance is made of a comfortable heat-sensitive acrylic and is reinforced by a metal framework. The adjustment mechanism (Herbst-type) is positioned on the sides of the device and allows good freedom of movement of the jaw. This appliance is a frequent choice for patients who tend to grind their teeth heavily. Adjustable pm positioner; Dorsal (somnodent); Dream tap; Ema; Herbst; Micro2; Narval; Suad are some mandibular (jaw) advancement appliances which allows the freedom for good movement of jaw.

The inefficiency of the above appliances is that they can only be applied on ages 7-11 years old. If they are not started in between age 7-1, they can not be done on people. The deficiencies are that the age for treatment is very limited to age 7-11 yrs old only. Also,it flares the upper teeth way out, and forces the lower jaw forward. Most of the sleep appliances and MAD appliances are night time only appliances and do not permanently change or develop the jaw structures. They help to open the airway during sleeping only at night.

The following are the drawbacks of the prior arts:
1. Some of these products that develop the upper and lower jaws forward can only be done after age 6 when the first permanent molars are fully erupted into the mouth.
2. The age for treatment for some products is very limited to age 7-11 yrs old only. Also, treatment flares the upper teeth way out, and forces the lower jaw forward, which is a difficult and uncomfortable process.
3. Most of the sleep appliances and MAD appliances are nighttime only appliances and do not permanently change or develop the jaw structures. They help to open the airway during sleeping only at night by moving the lower jaw forward temporarily and there is a distalizing force on this upper jaw.
4. The oral sleep appliances support the lower jaw in a more forward position moving the back of the tongue forward as well, but the front of the tongue is still restricted by the front teeth of the upper jaw which is commonly underdeveloped.
5. There are no appliances that can be used to develop the upper and lower jaws forward in the all primary dentition (all baby teeth with no permanent teeth in yet, ages 3-6).
6. There are no appliances that can be used to develop the upper and lower jaws forward in the early primary dentition with only 16 baby teeth erupted into the mouth (ages 18 months to two years of age).
7. No prior art describes changing the vertical angle of the screw in precise increments to reverse excess vertical or downward growth.
8. No prior art describes embodiments that cover the canines to maintain the canine roots in a vertical orientation while developing the front of the upper jaw upward and forward in a controlled fashion.
9. No prior art describes a support wire to improve strength and durability of the removable acrylic appliances.
10. No prior art is able to predictable reverse a gummy smile or excess downward jaw growth non surgically in a large variety of age groups.

So, there is a need for a removable airway appliance that grows the jaw bone forward, guiding the lower jaw bone to grow forward with the upper jaw bone, getting the forward jaw growth of the upper and lower jaws, starting at any age, even at age 2 all the way to advanced adulthood. The present invention uses in all primary dentition (all baby teeth) and permanent teeth for people from age 2-to older adult and can be used in a one year old with only 16 baby teeth. The present invention has the ability to reverse excess vertical/downward growth (controlling the amount and direction of growth/development) and has the ability to guide growing jaws to improve airway (covering canines) or making room for teeth (acrylic behind canines). Smooth surface over the biting area of the teeth help to stimulate the forward growth of the lower jaw and creates a more open airway to breathe better and to reduce or eliminate sleep apnea. The present invention helps to grow and develop the jaw structures permanently in more forward and ideal position. The present invention helps develops the bone of the upper and lower jaws forward permanently.

Thus, the present disclosure provides the state of the art with a novel, removable airway appliance in the Maxilla, comprising an acrylic base molded over certain portions of the upper dentition wherein the base includes one or more screw adjustment members operable to translate a section of the base in an upward and/or forward direction, creating more space in the mouth for the tongue by developing the bone of the upper and lower jaws forward permanently and a method of use utilizing wire clasps may also be used to secure the device to the dentition. As more space is developed in the mouth for the tongue, there is also more space at the back of the throat for airflow and breathing because the tongue is in a more forward position.

### OBJECTIVES OF THE DISCLOSURE

The main objective of the present invention is to remove all the associated drawbacks and to provide a novel, a removable airway appliance for developing the bone of the upper and lower jaws forward permanently and a method of use.

Yet another object of the present invention is to guide and develop the lower and upper jaws forward, also widen the upper jaw, making room for the tongue in the mouth to maximize the airway space by the precise angulation of the screws.

Yet another object of the present invention, for young children ages 1-6, is to prevent crowding of the teeth and to make room for all permanent teeth to come into the mouth without crowding. Creating space for all the permanent teeth before they come in and prevent crowding, especially useful for young children to avoid future crowding.

Yet another object of the present invention is to develop the upper and lower jaws forward to get the back of the tongue forward away from the back of the throat which improves airflow for breathing and sleeping.

Yet another objective of the present invention is to help in growth of the the upper jaw forward and unrestrict the lower jaw that is often trapped back behind the upper jaw.

Another objective of the present invention is to provide a removable airway appliance which provides better airway, better nasal breathing, upper and lower jaw forward growth, provides better cheek structure and better lip support to the human face.

Another object of the present invention is to wear the appliance 24 hours a day only removing a few minutes a day to brush for younger children (age 1-13) and worn 22hours a day for older teenagers and adults age 14+.

Another object of the present invention is to Use the removable airway appliance in all primary dentition (all baby teeth) and permanent teeth for people from age 2-to older adult.

Another object of the present invention is that it can be used with only 16 baby teeth.

Another object of the present invention is to reverse excess vertical/downward growth (controlling the amount and direction of growth/development) by using the removable airway appliance by the vertical angulation of the screws. In this way it can help to reverse an excessively gummy smile.

Another object of the present invention is to control growing jaws to improve airway (covering canines) or making room for teeth (acrylic behind canines).

Another object of the present invention is to provide a Smooth surface over the biting area of the teeth and help to stimulate the forward growth of the lower jaw.

Another object of the present invention is to create a more open airway to breathe better and to reduce or eliminate sleep apnea.

Another object of the present invention is to expand (dental arches) the crescent arrangement of the patient's teeth. Even in cases of severe crowding in children or adults, no permanent teeth need to be removed to develop the jaws, open the airway and straighten the teeth.

### SUMMARY OF THE DISCLOSURE

The present disclosure provides the state of the art with a novel removable airway appliance for guiding and developing the lower and upper jaws forward. Furthermore, the present disclosure provides removable airway appliance for guiding and developing the lower and upper jaws forward. The present invention prevents crowding of the teeth and makes room for all the teeth, making room for all permanent teeth without crowding, creates space for all the permanent teeth. The present invention is that it may grow the upper jaw forward and unrestrict the lower jaw that is often trapped back behind the upper jaw. The present invention guides and develops the lower and upper jaws forward, and widen the upper jaw, making room for the tongue in the mouth to maximize the airway space. In removable airway appliance, on the horizontal axis the screws can be positioned flat at a small angle or at a steeper angle to help direct the direction of growth and development of the jaw bone. The side angle is directing the amount of increased width of the arch.

Systems that embody the disclosure, in accordance with the aspects thereof, are typically designed by describing their functions. Therefore, the present disclosure is also embodied in such descriptions, and methods of describing systems, but the scope of the present disclosure is not limited thereby. Therefore, these and other objects of the disclosure are hereby considered, not only those limited to those found in this summary of the disclosure section, but as can be found throughout the scope of this specification and accompanying documents.

### BRIEF DESCRIPTION OF THE DRAWINGS

The drawings 1 to 8 accompanying and forming part of this specification are included to depict certain aspects of the disclosure.

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural and other changes may be made without departing from the spirit and scope of the disclosure. The following detailed description is, therefore, not to be taken in a limiting sense, and the scope of the disclosure is defined only by the appended claims, along with the full scope of equivalents to which such claims are entitled.

The present invention is a removable airway appliance for developing jaw structure. The screws are set at an up/down angle and a side-side angle. The screws are set on the appliance, and it is for the primary dentition on the baby teeth, a second system for permanent teeth, and for those that have some baby teeth and some permanent teeth.

In one embodiment, the present invention is that it may grow the upper jaw forward and unrestrict the lower jaw that is often trapped back behind the upper jaw. The present invention guides and develops the lower and upper jaws forward, and widens the upper jaw, making room for the tongue in the mouth to maximize the airway space. The present removable airway appliance guides and makes room for all the teeth, making room for all permanent teeth without crowding. Parents who begin their young children with this treatment can create space for all the permanent teeth before they come in and prevent crowding. The present invention develops the upper and lower jaws forward that help get the back of the tongue forward away from the back of the throat which improves airflow for breathing and sleeping. The present invention develops the upper and lower jaws forward to get the back of the tongue forward away from the back of the throat which improves airflow for breathing and sleeping.The present invention helps in growth of the the upper jaw forward and unrestrict the lower jaw that is often trapped back behind the upper jaw. The present invention provides better airway, better nasal breathing, upper and lower jaw forward growth, provides better cheek structure and better lip support to the human face. The removable airway appliance can be worn 24 hours a day only removing a few minutes a day to brush for younger children (age 1-13) and worn 22 hours a day for older teenagers and adults age 14+. Removable airway appliance method uses the removable airway appliance in all primary dentition (all baby teeth) and permanent teeth for people from age 2-to older adult and it can be used with only 16 baby teeth.

The present invention reverses excess vertical/downward growth (controlling the amount and direction of growth/development) by using the removable airway appliance. The present invention controls growing jaws to improve airway (covering canines) or making room for teeth (acrylic behind canines) and provides a smooth surface over the biting area of the teeth and help to stimulate the forward growth of the lower jaw. The present invention creates a more open airway to breathe better and to reduce or eliminate sleep apnea.

The present invention uses in all primary dentition (all baby teeth) and permanent teeth for people from age 2-to older adult. The present invention is used with only 16 baby teeth and to reverse excess vertical/downward growth (controlling the amount and direction of growth/development). The present invention controls growing jaws to improve airway (covering canines) or making room for teeth (acrylic behind canines) and Smooth surface over the biting area of the teeth help to stimulate the forward growth of the lower jaw.

The present invention creates a more open airway to breathe better and to reduce or eliminate sleep apnea. For wearing the appliance, the occlusal thickness of the expander is assessed using calipers and the lowest cusp on either side is measured from left to right to ensure proper thickness. Any type of adjustments are carried out if needed. The expander is applied in patient's mouth. Some precaution are taken before application of the appliance like: acrylic is fully pressed up against the palate, the bite is checked to see if articulating paper holds on both sides for proper thickness. Care should be taken that the appliance is not rocking in the mouth when they bite down, when patient is hitting both sides evenly, when they bite down. Care should be taken that the patient is not feeling any pain. If any adjustments are needed, are done to acrylic for removal of sharp or rough edges and care should be taken that an even occlusal surface is there from left to right. Instructions are given to the guardian/adult person on how to turn the key hold expander with the section that is up against the roof of the mouth towards the floor. The expander is hold securely where the key is being inserted. This prevents pressure that could break the expander. The key is inserted into the hole and the key is turned in the direction of the arrow, towards the floor. Turning the key to a full 90. It is always a “downhill” direction to expand. Reset the expander back to the beginning and allow the guardian to turn the key to ensure they understand watching for which direction, which keyhole and that they are supporting the expander well

The steps for applying the removable airway appliance of the present invention are as follows: Up-down angle reversing excess vertical/downward growth. This gets upward and forward development. This vertical angle can be 0 degrees, 5 degrees, 10 degrees, 15 degrees, 20 degrees, 25 degrees, 30 degrees.

Up-down angle parallel to teeth for upward and forward development.

Passive retention for all baby teeth and clasping system when permanent teeth come in.

Side angle used to control the amount of lateral/sideways growth and development.

Acrylic covering palate and back of front teeth to help control forces of forward/upward growth and development.

With an edge to edge or open bite, there is no overbite, the upper teeth have not come too far downward. There is not a gummy smile. However, the jaws still need to be developed forward. Developing the upper and lower jaws forward will give an improved airway for improved breathing and sleeping. The screws are positioned at a zero degree angle the direction of growth and development is in the forward direction. Again, this angle can be zero degrees to thirty degrees, using a higher degree angle when we want to correct a severe overbite, an excess of vertical growth or a gummy smile.

In another embodiment, the present invention has the retention through the complete coverage of the back teeth/back molars, for all primary/all baby teeth. There are no hooks or metal clasps hooking to the teeth to hold it in place. When permanent teeth are in then the retention is from the metal “hang clasps” that hook onto two of the back teeth on each side. The two metal hooks shown are the clasps that are on two back teeth on each side of the upper teeth. Another aspect is the amount of acrylic covering the palate/roof of the mouth and the lingual side/back side of the upper front teeth. When we want to change the vertical or up/down position of the front of the upper jaw we have less acrylic on the palate and more covering the back side of the upper teeth. When we want forward growth at zero or 5 degrees, we have the acrylic wider on the front palate/roof of the mouth and no acrylic on the back side of the upper front teeth. Anterior acrylic section covers a specified amount of the palate/roof of the mouth and lingual side/inside of top front teeth to direct the forces of development.

In Combination, these unique features can combine in different ways for different applications such as for; severe overbite, moderate overbite, slight overbite, open bite, Class I, II, or III with Maxillary deficiency, and narrow or wide jaws. When the side-to-side position of the screws are set, they can be placed parallel for no lateral or no sideways development and only forward development. The side-to-side angle/position of the screws can be placed at as light angle to get increased width of the molars and a wider mouth as the forward growth is developed. The side-to-side angle of the screws can be put at a much steeper angle in relation to each other. This would be used for a very narrow mouth and achieve significant increased width or widening and lateral or sideways development of the mouth.

In case, when the mouth is very wide already and for forward development, the screws are placed parallel. When the mouth is average width, the screws are placed at a slight angle for gradual increase in width. When the mouth is very narrow, the screws are placed at an angle to increase the width faster.

In another embodiment, the removable airway appliance is “U” shaped appliance having two protruded portions on the front side. The removable airway appliance comprising of
a. Anterior/Front acrylic section having a specified amount of the palate/roof of the mouth and lingual side/inside of top front teeth to direct the forces of development and a support wire embedded in the acrylic for added strength.
b. Posterior/back acrylic section providing retention over the primary /baby molars.
c. Expansion screws set at different angles for up/down and side/side growth and development having full coverage over canines.
d. Hang clasps (for retention (in case if permanent teeth are present) including (front clasp and back clasp)
e. Front support wire screw at 30°.

In another embodiment, in removable airway appliance, on the horizontal axis the screws can be positioned flat at a small angle or at a steeper angle to help direct the direction of growth and development of the jawbone. The side angle is directing the amount of increased width of the arch. Appliances are worn 24 hours a day only removing a few minutes a day to brush for younger children (age 1-13) and worn 22 hours a day for older teenagers and adults age 14+.

The screw assembly is fabricated of acrylic alloys. The front and back section are fabricated from acrylic. There is a complete acrylic coverage over the primary molars.

Both sides are connected by front support wire which maintains the desired lateral separation between front section and back sect.

In another embodiment, 1. Stage 1 Early Primary Dentition Open Bite (in which Front teeth do not touch), Narrow Mouth Embodiment: 16 primary teeth, complete acrylic coverage over the primary first molar and primary canine. Acrylic behind the upper front teeth, screws parallel to teeth and ridge, screws at a 0 degree vertical angle (straight).
Stage 1 Early Primary Dentition Slight Overbite (1-30% Overbite), Narrow Mouth Embodiment: 16 primary teeth, complete acrylic coverage over the primary first molar and primary canine. Acrylic behind the upper front teeth, screws parallel to teeth and ridge, screws at a 10 degree angle upward.
Stage 1 Early Primary Dentition Moderate Overbite (31-65% Overbite), Narrow Mouth Embodiment: 16 primary teeth, complete acrylic coverage over the primary first molar and primary canine. Acrylic behind the upper front teeth, screws parallel to teeth and ridge, screws at a 20 degree angle upward.
Stage 1 Early Primary Dentition Deep Bite (66-100% Overbite), Narrow Mouth Embodiment: 16 primary teeth, complete acrylic coverage over the primary first molar and primary canine. Acrylic behind the upper front teeth, screws parallel to teeth and ridge, screws at a 30 degree angle upward.
Stage 1 Early Primary Dentition Open Bite (Front teeth do not touch), Wide Mouth Embodiment: 16 primary teeth, complete acrylic coverage over the primary first molar and primary canine. Acrylic behind the upper front teeth, screws parallel to each other, screws at a 30 degree angle upward.
Stage 1 Early Primary Dentition Slight Overbite (1-30% Overbite), Wide Mouth Embodiment: 16 primary teeth, complete acrylic coverage over the primary first molar and primary canine. Acrylic behind the upper front teeth, screws parallel to each other, screws at a 30 degree angle upward.
Stage 1 Early Primary Dentition Moderate Overbite (31-65% Overbite), Wide Mouth Embodiment: 16 primary teeth, complete acrylic coverage over the primary first molar and primary canine. Acrylic behind the upper front teeth, screws parallel to each other, screws at a 30 degree angle upward.
Stage 1 Early Primary Dentition Deep Bite (66-100% Overbite), Wide Mouth Embodiment: 16 primary teeth, complete acrylic coverage over the primary first molar and primary canine. Acrylic behind the upper front teeth, screws parallel to each other, screws at a 30 degree angle upward.
Stage 1 Primary Dentition Open Bite (Front teeth do not touch), Narrow Mouth Embodiment: 20 primary teeth, complete acrylic coverage over the primary molars. Acrylic behind the upper front teeth, screws parallel to teeth and ridge, screws at a 0 degree vertical angle (straight).
Stage 1 Primary Dentition Slight Overbite (1-30% Overbite), Narrow Mouth Embodiment: 20 primary teeth, complete acrylic coverage over the primary molars. Acrylic behind the upper front teeth, screws parallel to teeth and ridge, screws at a 10 degree angle upward.
Stage 1 Primary Dentition Moderate Overbite (31-65% Overbite), Narrow Mouth Embodiment: 20 primary teeth, complete acrylic coverage over the primary molars. Acrylic behind the upper front teeth, screws parallel to teeth and ridge, screws at a 20 degree angle upward.
Stage 1 Primary Dentition Deep Bite (66-100% Overbite), Narrow Mouth Embodiment: 20 primary teeth, complete acrylic coverage over the primary molars. Acrylic behind the upper front teeth, screws parallel to teeth and ridge, screws at a 30 degree angle upward.
Stage 1 Primary Dentition Open Bite (Front teeth do not touch), Wide Mouth Embodiment: 20 primary teeth, complete acrylic coverage over the primary molars. Acrylic behind the upper front teeth, screws parallel to each other, screws at a 30 degree angle upward.
Stage 1 Primary Dentition Slight Overbite (1-30% Overbite), Wide Mouth Embodiment: 20 primary teeth, complete acrylic coverage over the primary molars. Acrylic behind the upper front teeth, screws parallel to each other, screws at a 30 degree angle upward.
Stage 1 Primary Dentition Moderate Overbite (31-65% Overbite), Wide Mouth Embodiment: 20 primary teeth, complete acrylic coverage over the primary molars. Acrylic behind the upper front teeth, screws parallel to each other, screws at a 30 degree angle upward.
Stage 1 Primary Dentition Deep Bite (66-100% Overbite), Wide Mouth Embodiment: 20 primary teeth, complete acrylic coverage over the primary molars. Acrylic behind the upper front teeth, screws parallel to each other, screws at a 30 degree angle upward.
Stage 1 Mixed Dentition Open Bite (Front teeth do not touch), Narrow Mouth Embodiment: 20 primary teeth, complete acrylic coverage over the primary molars. Acrylic behind the upper front teeth, screws parallel to teeth and ridge, screws at a 0 degree vertical angle (straight).
Stage 1 Mixed Dentition Slight Overbite (1-30% Overbite), Narrow Mouth Embodiment: 20 primary teeth, complete acrylic coverage over the primary molars. Acrylic behind the upper front teeth, screws parallel to teeth and ridge, screws at a 10 degree angle upward.
Stage 1 Mixed Dentition Moderate Overbite (31-65% Overbite), Narrow Mouth Embodiment: 20 primary teeth, complete acrylic coverage over the primary molars. Acrylic behind the upper front teeth, screws parallel to teeth and ridge, screws at a 20 degree angle upward.
Stage 1 Mixed Dentition Deep Bite (66-100% Overbite), Narrow Mouth Embodiment: 20 primary teeth, complete acrylic coverage over the primary molars. Acrylic behind the upper front teeth, screws parallel to teeth and ridge, screws at a 30 degree angle upward.
Stage 1 Mixed Dentition Open Bite (Front teeth do not touch), Wide Mouth Embodiment: 20 primary teeth, complete acrylic coverage over the primary molars. Acrylic behind the upper front teeth, screws parallel to each other, screws at a 30 degree angle upward.
Stage 1 Mixed Dentition Slight Overbite (1-30% Overbite), Wide Mouth Embodiment: 20 primary teeth, complete acrylic coverage over the primary molars. Acrylic behind the upper front teeth, screws parallel to each other, screws at a 30 degree angle upward.
Stage 1 Mixed Dentition Moderate Overbite (31-65% Overbite), Wide Mouth Embodiment: 20 primary teeth, complete acrylic coverage over the primary molars. Acrylic behind the upper front teeth, screws parallel to each other, screws at a 30 degree angle upward.
Stage 1 Mixed Dentition Deep Bite (66-100% Overbite), Wide Mouth Embodiment: 20 primary teeth, complete acrylic coverage over the primary molars. Acrylic behind the upper front teeth, screws parallel to each other, screws at a 30 degree angle upward.
Stage 1 Permanent Dentition Open Bite (Front teeth do not touch), Narrow Mouth Embodiment: 20 primary teeth, complete acrylic coverage over the primary molars. Acrylic behind the upper front teeth, screws parallel to teeth and ridge, screws at a 0 degree vertical angle (straight).
Stage 1 Permanent Dentition Slight Overbite (1-30% Overbite), Narrow Mouth Embodiment: 20 primary teeth, complete acrylic coverage over the primary molars. Acrylic behind the upper front teeth, screws parallel to teeth and ridge, screws at a 10 degree angle upward.
Stage 1 Permanent Dentition Moderate Overbite (31-65% Overbite), Narrow Mouth Embodiment: 20 primary teeth, complete acrylic coverage over the primary molars. Acrylic behind the upper front teeth, screws parallel to teeth and ridge, screws at a 20 degree angle upward.
Stage 1 Permanent Dentition Deep Bite (66-100% Overbite), Narrow Mouth Embodiment: 20 primary teeth, complete acrylic coverage over the primary molars. Acrylic behind the upper front teeth, screws parallel to teeth and ridge, screws at a 30 degree angle upward.
Stage 1 Permanent Dentition Open Bite (Front teeth do not touch), Wide Mouth Embodiment: 20 primary teeth, complete acrylic coverage over the primary molars. Acrylic behind the upper front teeth, screws parallel to each other, screws at a 30 degree angle upward.
Stage 1 Permanent Dentition Slight Overbite (1-30% Overbite), Wide Mouth Embodiment: 20 primary teeth, complete acrylic coverage over the primary molars. Acrylic behind the upper front teeth, screws parallel to each other, screws at a 30 degree angle upward.
Stage 1 Permanent Dentition Moderate Overbite (31-65% Overbite), Wide Mouth Embodiment: 20 primary teeth, complete acrylic coverage over the primary molars. Acrylic behind the upper front teeth, screws parallel to each other, screws at a 30 degree angle upward.
Stage 1 Permanent Dentition Deep Bite (66-100% Overbite), Wide Mouth Embodiment: 20 primary teeth, complete acrylic coverage over the primary molars. Acrylic behind the upper front teeth, screws parallel to each other, screws at a 30 degree angle upward.

In another embodiment,
Stage 2 Primary Dentition Open Bite (Front teeth do not touch), Narrow Mouth Embodiment: 20 primary teeth, complete acrylic coverage over the primary molars. Acrylic completely over the upper canines and behind the other upper front teeth, screws parallel to teeth and ridge, screws at a 0 degree vertical angle (straight).
Stage 2 Primary Dentition Slight Overbite (1-30% Overbite), Narrow Mouth Embodiment: 20 primary teeth, complete acrylic coverage over the primary molars. Acrylic completely over the upper canines and behind the other upper front teeth, screws parallel to teeth and ridge, screws at a 10 degree angle upward.
Stage 2 Primary Dentition Moderate Overbite (31-65% Overbite), Narrow Mouth Embodiment: 20 primary teeth, complete acrylic coverage over the primary molars. Acrylic completely over the upper canines and behind the other upper front teeth, screws parallel to teeth and ridge, screws at a 20 degree angle upward.
Stage 2 Primary Dentition Deep Bite (66-100% Overbite), Narrow Mouth Embodiment: 20 primary teeth, complete acrylic coverage over the primary molars. Acrylic completely over the upper canines and behind the other upper front teeth, screws parallel to teeth and ridge, screws at a 30 degree angle upward.
Stage 2 Primary Dentition Open Bite (Front teeth do not touch), Wide Mouth Embodiment: 20 primary teeth, complete acrylic coverage over the primary molars. Acrylic completely over the upper canines and behind the other upper front teeth, screws parallel to each other, screws at a 30 degree angle upward.
Stage 2 Primary Dentition Slight Overbite (1-30% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.
Stage 2 Primary Dentition Moderate Overbite (31-65% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.
Stage 2 Primary Dentition Deep Bite (66-100% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.
Stage 2 Mixed Dentition Open Bite (Front teeth do not touch), Narrow Mouth Embodiment: 20 primary teeth, complete acrylic coverage over the primary molars. Acrylic completely over the upper canines and behind the other upper front teeth, screws parallel to teeth and ridge, screws at a 0 degree vertical angle (straight).
Stage 2 Mixed Dentition Slight Overbite (1-30% Overbite), Narrow Mouth Embodiment: 20 primary teeth, complete acrylic coverage over the primary molars. Acrylic completely over the upper canines and behind the other upper front teeth, screws parallel to teeth and ridge, screws at a 10 degree angle upward.
Stage 2 Mixed Dentition Moderate Overbite (31-65% Overbite), Narrow Mouth Embodiment: 20 primary teeth, complete acrylic coverage over the primary molars. Acrylic completely over the upper canines and behind the other upper front teeth, screws parallel to teeth and ridge, screws at a 20 degree angle upward.
Stage 2 Mixed Dentition Deep Bite (66-100% Overbite), Narrow Mouth Embodiment: 20 primary teeth, complete acrylic coverage over the primary molars. Acrylic completely over the upper canines and behind the other upper front teeth, screws parallel to teeth and ridge, screws at a 30 degree angle upward.
Stage 2 Mixed Dentition Open Bite (Front teeth do not touch), Wide Mouth Embodiment: 20 primary teeth, complete acrylic coverage over the primary molars. Acrylic completely over the upper canines and behind the other upper front teeth, screws parallel to each other, screws at a 30 degree angle upward.
Stage 2 Mixed Dentition Slight Overbite (1-30% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.
Stage 2 Mixed Dentition Moderate Overbite (31-65% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.
Stage 2 Mixed Dentition Deep Bite (66-100% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.
Stage 2 Permanent Dentition Open Bite (Front teeth do not touch), Narrow Mouth Embodiment: 20 primary teeth, complete acrylic coverage over the primary molars. Acrylic completely over the upper canines and behind the other upper front teeth, screws parallel to teeth and ridge, screws at a 0 degree vertical angle (straight).
Stage 2 Permanent Dentition Slight Overbite (1-30% Overbite), Narrow Mouth Embodiment: 20 primary teeth, complete acrylic coverage over the primary molars. Acrylic completely over the upper canines and behind the other upper front teeth, screws parallel to teeth and ridge, screws at a 10 degree angle upward.
Stage 2 Permanent Dentition Moderate Overbite (31-65% Overbite), Narrow Mouth Embodiment: 20 primary teeth, complete acrylic coverage over the primary molars. Acrylic completely over the upper canines and behind the other upper front teeth, screws parallel to teeth and ridge, screws at a 20 degree angle upward.
Stage 2 Permanent Dentition Deep Bite (66-100% Overbite), Narrow Mouth Embodiment: 20 primary teeth, complete acrylic coverage over the primary molars. Acrylic completely over the upper canines and behind the other upper front teeth, screws parallel to teeth and ridge, screws at a 30 degree angle upward.
Stage 2 Permanent Dentition Open Bite (Front teeth do not touch), Wide Mouth Embodiment: 20 primary teeth, complete acrylic coverage over the primary molars. Acrylic completely over the upper canines and behind the other upper front teeth, screws parallel to each other, screws at a 30 degree angle upward.
Stage 2 Permanent Dentition Slight Overbite (1-30% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.
Stage 2 Permanent Dentition Moderate Overbite (31-65% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.
Stage 2 Permanent Dentition Deep Bite (66-100% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.

The removable appliance can be taken out, the patient comes in for simple adjustments every four to twelve weeks. The upper arch expands anteriorly (to the front) by approximately 1 mm a month as can be seen by the pictures below. Once the desired spaces are achieved between the upper canines and the first primary molars behind, the appliance is removed and a retainer is inserted inside the mouth to avoid relapse, to make sure that the teeth do not collapse back to the original position. After the permanent teeth come in the bone is stable. Braces may not be needed in all cases. If needed, braces are placed on both upper and lower teeth. The braces with the help of elastic bands moves the back teeth forward to close the gaps, while maintaining the position of the front teeth. This procedure enhances the chance of all wisdom teeth erupting in the correct position, without the need for extractions or headgear.

In the meantime, the lower braces can straighten the lower teeth if needed. Once all the gaps are closed, the upper and lower teeth are coordinated to ensure optimal aesthetics and function.

The end result is an expanded upper jaw both towards the front and the sides. The jaw is able to accommodate all permanent teeth (may include wisdom teeth if the conditions are right) to erupt in the correct position, the lower jaw is able to occlude into the ideal bite in harmony with the TMJ joints and the facial muscles. The ensuing enlarged space in the mouth allows the tongue to position itself correctly against the roof of the mouth, opening good airways at the back of the throat and inside the nose. The removable appliance is designed for children to accommodate the orofacial growth. The appliance is turned (activated) twice a week by the patients themselves, or for the very young ones, by their parents. It is to be worn 24/7 including eating. The appliance expands the upper jaw in all dimensions, to lay down the foundation and to accommodate the eruption of the incoming permanent teeth. When the permanent teeth are ready to erupt down there will be adequate amount of space.

The working of the removable airway appliance is as follows.

The removable airway appliance is attached to back teeth and gently, pressure is put on the nasopalatine nerve that runs through palate as well as right forward pressure on the bone and the canines and front teeth stimulating bone remodeling and development. This pressure stimulates growth in the upper jaw, which also allows the lower jaw to develop forward. When this growth takes place, it can create room for even severely crowded teeth to align, and it can open up the airway, enlarge your airway and move the mid and lower face forward. The present invention helps to expand the jaws forward without the need for surgery. More benefits can include long-term relief from issues like TMD pain and Sleep Apnea without the long-term use of oral appliances. The present invention helps in sleep apnea and snoring because when a person undergoes the maxillary expansion of jaw, the airway can be opened. And when upper jaw expands during this treatment process, it can effectively realign the lower jaw, helping to relieve TMD clicking, popping and pain without surgery. This happens because as the upper and lower jaws develop forward, there is more space for the disc of the TMJ to function smoothly.

Up-down angle parallel to teeth for upward and forward development. Up-down angle reverses excess vertical/downward growth. This gets upward and forward development. This angle can be 0 degrees, 5 degrees, 10 degrees, 15 degrees, 20 degrees, 25 degrees, 30 degrees. The side angle used to control the amount of lateral/sideways growth and development. Acrylic covering palate and back of front teeth to help control forces of forward/upward growth and development.

Advantages of the present invention:

The present invention uses in all primary dentition (all baby teeth) and permanent teeth for people from age 2-to older adult and Can be used not only in the all primary dentition, but also with only 16 baby teeth. The present invention has the Ability to reverse excess vertical/downward growth (controlling the amount and direction of growth/development) and has the ability to control growing jaws to improve airway (covering canines) or making room for teeth (acrylic behind canines). Smooth surface over the biting area of the teeth help to stimulate the forward growth of the lower jaw and creates a more open airway to breathe better and to reduce or eliminate sleep apnea. The present invention helps to grow and develop the jaw structures permanently in more forward and ideal position. The present invention helps develops the bone of the upper and lower jaws forward permanently. It can non-surgically develop the Maxilla in an upward direction reversing a gummy smile.

1. A “U” shaped removable airway appliance (100) having two protruded portions (8) on the front side, for reverse excessing vertical/downward growth and developing the upper and lower jaws bones permanently in a forward position, comprising of;
a. Anterior/Front acrylic section having a specified amount of the palate/roof of the mouth and lingual side/inside of top front teeth to direct the forces of development (1.1);
b. a support wire embedded in the acrylic for added strength (1.2);
c. Posterior/back acrylic section (2) providing retention over the primary /baby molars (3);
d. Expansion screws set (4) at different angles for up/down and side/side growth and development having full coverage over canines (5);
e. Hang clasps (6) (for retention (in case if permanent teeth are present) including (front clasp (6.1) and back clasp (6.2));
f. Front support wire screw at 30√ (7).

2. The “U” shaped removable airway appliance (100) having two protruded portions on the front side, having two protruded portions (8) on the front side, for reverse excessing vertical/downward growth and developing the upper and lower jaws bones permanently in a forward position, as claimed in claim 1, wherein the said appliance (100) uses in all primary dentition (all baby teeth) and permanent teeth for people from age 2-to older adult and can be used in a one year old with only 16 baby teeth.

3. The “U” shaped removable airway appliance (100) having two protruded portions on the front side, having two protruded portions (8) on the front side, for reverse excessing vertical/downward growth and developing the upper and lower jaws bones permanently in a forward position, as claimed in claim 1, wherein the said the occlusal thickness of the expander (9) is assessed using calipers and the lowest cusp on either side is measured from left to right to ensure proper thickness.

4. The “U” shaped removable airway appliance (100) having two protruded portions on the front side, having two protruded portions (8) on the front side, for reverse excessing vertical/downward growth and developing the upper and lower jaws bones permanently in a forward position, as claimed in claim 1, wherein the said expander is applied in patient's mouth and hold securely where the key is being inserted into the hole and the key is turned in the direction of the arrow, towards the floor. Turning the key to a full 90.

5. The “U” shaped removable airway appliance (100) having two protruded portions on the front side, having two protruded portions (8) on the front side, for reverse excessing vertical/downward growth and developing the upper and lower jaws bones permanently in a forward position, as claimed in claim 1, wherein the said screws are positioned at a zero degree to thirty degrees, using a higher degree angle If the direction of growth and development is in the forward direction.

6. The “U” shaped removable airway appliance (100) having two protruded portions on the front side, having two protruded portions (8) on the front side, for reverse excessing vertical/downward growth and developing the upper and lower jaws bones permanently in a forward position, as claimed in claim 1, wherein the said appliance is combined in different ways for different applications such as for; severe overbite, moderate overbite, slight overbite, open bite, Class I, II, or III with Maxillary deficiency, and narrow or wide jaws.

7. The “U” shaped removable airway appliance (100) having two protruded portions on the front side, having two protruded portions (8) on the front side, for reverse excessing vertical/downward growth and developing the upper and lower jaws bones permanently in a forward position, as claimed in claim 1, wherein the said Appliance is worn 24 hours a day (only removing a few minutes a day to brush) for younger children (age 1-13) and worn 22 hours a day for older teenagers and adults age 14+.

8. A process for application of the removable airway appliance (100) comprising the steps of;
a. reversing Up-down angle at 0 degrees, 5 degrees, 10 degrees, 15 degrees, 20 degrees, 25 degrees, 30 degrees for excess vertical/downward growth;
b. resetting Up-down angle parallel to teeth for upward and forward development;
c. retention of the Passive retention for all baby teeth and clasping system when permanent teeth come in;
d. controlling the amount of lateral/sideways growth using Side angle for development;
e. helping Acrylic covering palate and back of front teeth for controlling forces of forward/upward growth and development.
